(19) United States
(12) Patent Application Publication
(10) Pub. No.: US 2014/0288518 A1
(43) Pub. Date: Sep. 25, 2014

(54) Elastic Securement Using Foamed Hot Melt Adhesive in Absorbent Products

(71) Applicant: Associated Hygienic Products, LLC, Duluth, GA (US)

(72) Inventors: Trent Ottery, Delaware, OH (US); John Gladwin, Whitney, TX (US)

(73) Assignee: Associated Hygienic Products, LLC, Duluth, GA (US)

(21) Appl. No.: 14/211,993

(22) Filed: Mar. 14, 2014

Related U.S. Application Data
(60) Provisional application No. 61/792,739, filed on Mar. 15, 2013.

(51) Int. Cl. A61F 13/539

(52) U.S. Cl. CPC A61F 13/539; A61F 2013/53908
USPC 604/365

(57) ABSTRACT

Disposable absorbent articles comprising foamed adhesive for securing elastics are disclosed. In one embodiment, a disposable absorbent diaper is disclosed that comprises an adhesive that has been foamed by incorporating gas into a molten hotmelt adhesive. The resulting foamed adhesive displays increased surface area and volume and decreased density while retaining the same or about the same adhesive strength.

### PRIORITY CLAIM

This application claims priority to U.S. Provisional Application No. 61/792,739, filed Mar. 15, 2013.

### BACKGROUND OF THE INVENTION

1. FIELD OF THE INVENTION

The present invention relates to absorbent articles, and specifically to adhesives used in manufacturing absorbent articles.

2. BACKGROUND

Current elastic securement methods in absorbent products use solid slot coatings of hot melt adhesive or various solid spiral wrapping methods for adhering the elastic strand to a nonwoven substrate. Current adhesive materials technology has produced adhesives with much higher adhesive strength, but adhesive application methods have not kept up with these advances. As a result, adhesive for elastic securement in absorbent products is regularly applied in excess. Excessive adhesive application results in higher absorbent product production costs which are passed on to the consumer. There is a need in the industry for reducing the amount of adhesive required for adhering elastics to nonwoven substrates which preserves adhesive volume and strength.

The preferred embodiments discussed below seek to address some of these disadvantages in the prior art.

### SUMMARY OF THE INVENTION

Embodiments of the invention are disclosed that relate foamed hotmelt adhesives for securing elastics in absorbent products.

Foamed adhesive is made by adding a gas to a liquefied adhesive, which is then applied to an absorbent article.

Compared to conventional adhesives, foamed adhesives have a much lower density. Therefore, at an equivalent add-on by weight a foamed adhesive will have more volume and therefore increased surface contact with the elastic strands. This increase in volume can also add more loft in the z-direction (that is, the direction normal to the plane of an absorbent article) and stand up more than conventional adhesives do. The additional loft in the z-direction provides improved securement of elastic members.

Further, in certain ranges of gas-adhesive mixtures, foamed adhesives perform at least as well as conventional adhesives. This can reduce the overall amount of adhesive required for elastic securement.

In one embodiment, a disposable absorbent article is provided comprising: a topsheet; a backsheet; an absorbent core disposed between the topsheet and the backsheet; a front waist region; a back waist region; a crotch region; a plurality of elastic members adhered to the backsheet with a foamed hotmelt adhesive; where the foamed hotmelt adhesive comprises a hotmelt adhesive mixed with a gas such that the foamed hotmelt adhesive has a density that is reduced between about 30% to about 50% compared to the conventional adhesive without foaming.

In specific embodiments, the foamed hotmelt adhesive is between about 40% foamed and about 45% foamed, meaning that the density of the conventional adhesive has been reduced between about 40% to about 45%. In other words, the foamed adhesive is between about 40% to about 45% gas and about 60% to about 55% adhesive.

In certain embodiments, the gas is an inert gas such as nitrogen, while in other embodiments, the gas is air.

In various embodiments, some of the plurality of elastic members may be adhered to the backsheet at the crotch region, some may be adhered to the backsheet at the front waist region, and some may be adhered to the backsheet at the back waist region.

In another embodiment, a method of securing elastic members to a substrate with foamed hotmelt adhesive is presented, comprising introducing a gas to a molten hotmelt adhesive, and adhering elastic strands to the substrate via the foamed hotmelt adhesive.

### BRIEF DESCRIPTION OF THE DRAWINGS

The following drawings illustrate by way of example and not limitation. For the sake of brevity and clarity, every feature of a given structure may not be labeled in every figure in which that structure appears. Identical reference numbers do not necessarily indicate an identical structure. Rather, the same reference number may be used to indicate a similar feature or a feature with similar functionality, as may non-identical reference numbers.

FIG. 1 shows an embodiment of a disposable absorbent garment.

FIG. 2 shows a side section view of an embodiment of a disposable absorbent garment.

FIG. 3 shows an embodiment of a method for applying foamed adhesive.

### DETAILED DESCRIPTION

Various features and advantageous details are explained more fully with reference to the nonlimiting embodiments that are illustrated in the accompanying drawings and detailed in the following description. It should be understood, however, that the detailed description and the specific examples, while indicating embodiments of the invention, are given by way of illustration only, and not by way of limitation. Various substitutions, modifications, additions, and/or rearrangements within the spirit and/or scope of the underlying inventive concept will become apparent to those of ordinary skill in the art from this disclosure.

In the following description, numerous specific details are provided to provide a thorough understanding of the present embodiments. One of ordinary skill in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

The term “coupled” is defined as connected, although not necessarily directly, and not necessarily mechanically.

The terms “a” and “an” are defined as one or more unless this disclosure explicitly requires otherwise.

The term “substantially” and its variations (e.g. “approximately” and “about”) are defined as being largely but not necessarily wholly what is specified (and include wholly what is specified) as understood by one of ordinary skill in the art. In any embodiment of the present disclosure, the terms “substantially,” “approximately,” and “about” may be substituted with “within [a percentage] of” what is specified, where the percentage includes 0.1, 1, 5, 10, and 15 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements possesses those one or more steps or elements, but is not limited to possessing only those one or more elements. Likewise, a step of a method or an element of a device that “comprises,” “has,” “includes” or “contains” one or more features possesses those one or more features, but is not limited to possessing only those one or more features. Furthermore, a device or structure that is configured in a certain way is configured in at least that way, but may also be configured in ways that are not listed. Metric units may be derived from the English units provided by applying a conversion and rounding to the nearest millimeter.

The term “foamed,” when used with a percentage, refers to the density reduction of an adhesive that has been mixed with a gas compared to the density of the same adhesive that has not been mixed with a gas. For example, a hotmelt adhesive with a density of 1.0 grams per square meter (gsm) that is mixed with nitrogen gas to reduce its density to 0.6 gsm is a 40% foamed adhesive because the density of the adhesive has been reduced by 40%.

The feature or features of one embodiment may be applied to other embodiments, even though not described or illustrated, unless expressly prohibited by this disclosure or the nature of the embodiments.

Any embodiment of any of the present devices and methods can consist of or consist essentially of—rather than comprise/include/contain/have—any of the described elements and/or features and/or steps. Thus, in any of the claims, the term “consisting of” or “consisting essentially of” can be substituted for any of the open-ended linking verbs recited above, in order to change the scope of a given claim from what it would otherwise be using the open-ended linking verb.

Other features and associated advantages will become apparent with reference to the following detailed description of specific embodiments in connection with the accompanying drawings.

The discussion and figures below show a baby diaper for illustrative purposes only and are not intended to limit the scope of the invention. The disclosed absorbent cores can be used in any absorbent article, including training pants, adult incontinence products, and feminine hygiene products.

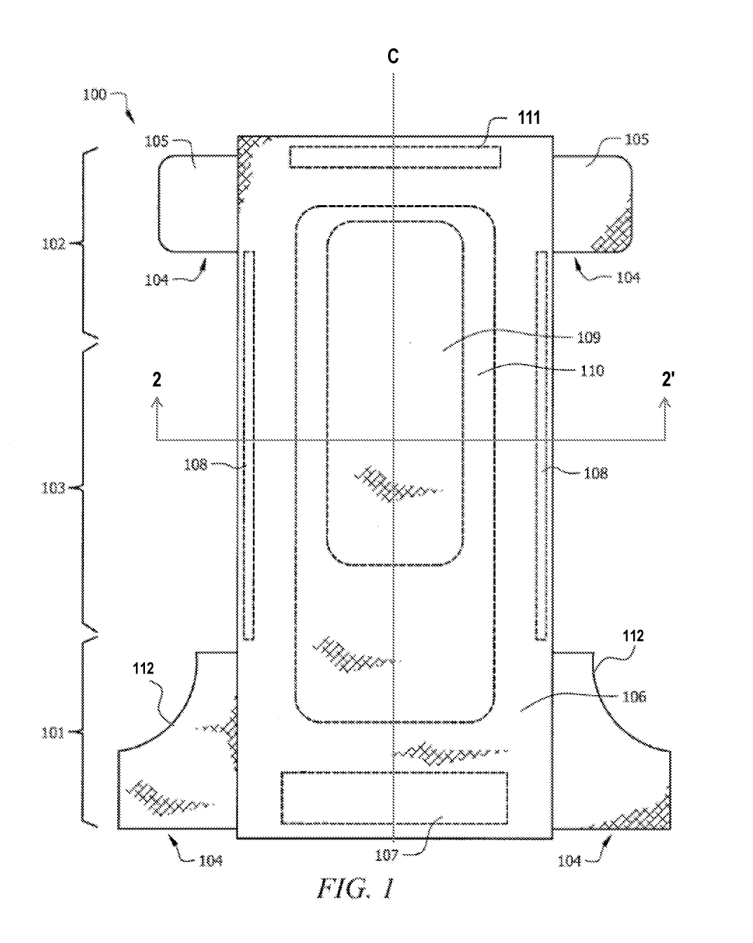

FIG. 1 illustrates an embodiment of a disposable absorbent garment 100. The disposable absorbent garment 100 is of a type that can be placed against or in proximity to the body of a wearer so as to absorb and contain various bodily exudates. The embodiment of FIG. 1 is introduced to illustrate some basic features of a disposable diaper, most of which are also applicable to other disposable absorbent garments contemplated by the present application. Garment 100 is shown in the unfastened configuration. Disposable garment 100 includes three main regions aligned along longitudinal centerline C. These regions include a first waist region 101, typically located at the back of the user when the garment is worn, a front waist region 102, and a crotch region 103. The shape of garment 100 may vary, and includes a generally rectangular shape, a T style shape, an I style shape, an hourglass shape, a combination of these shapes, or the like.

Disposable garment 100, at the back 101 and front 102 waist regions may include ear regions 104 comprising back panels 112 (sometimes known as side panels) extending generally perpendicular to the longitudinal centerline at waist regions 101 and 102. In this way, the back 101 and front 102 waist regions may be brought together and attached at the ear region to form holes to accommodate the wearer's legs. The shapes and configurations of ear portions 104 may be formed in any manner to accomplish the particular purposes of disposable garment 100. For example, back panels 112 may be configured to be permanently or refastenably attached.

Disposable garment 100 also comprises fastener means 105 to attach the back 101 and front 102 waist portions of the garment together, to form an opening for the leg of the wearer. These fasteners 105 may be integrated onto the backsheet or topsheet 106, or may be attached to the topsheet or backsheet (not shown) as additional tabs. Examples of fasteners 105 may include hook and loop fasteners, buttons, pins, snaps, adhesive tape fasteners, cohesives, mushroom and loop fasteners, or the like.

More specifically, loop type fasteners typically comprise a fabric or material having a base or backing structure and a plurality of loop members extending upwardly from at least one surface of the backing structure. The loop material can be formed of any suitable material, such as acrylic, nylon or polyester, and can be formed by methods such as warp knitting, stitch bonding or needle punching. Suitable loop materials are known in the art and are available, for example, from manufacturers such as 3M, St. Paul Minn. (designated as a KN series loop). The loop material can be an integral portion of the outer surface or inner surface of the side panel rather than a separated material or structure.

Hook type fasteners typically comprise a fabric or material having a base or backing structure and a plurality of hook members extending upwardly from at least one surface of the backing structure. In contrast to the loop type fasteners which desirably comprise a flexible fabric, the hook material advantageously comprises a resilient material to minimize unintentional disengagement of the fastener components as a result of the hook material becoming deformed and catching on clothing or other items. The term “resilient” as used herein refers to an interlocking material having a predetermined shape and the property of the interlocking material to resume the predetermined shape after being engaged and disengaged from a mating, complementary interlocking material. Suitable hook material can be molded or extruded of nylon, polypropylene or another suitable material. Suitable single-sided hook materials for fasteners 105 are known in the art and are available, for example, from manufacturers such as 3M, St. Paul Minn. (designated as a CPL series loop).

Fasteners 105 may be located at the back 101 or front 102 waist region of garment 100, or at both. For example, in the representative embodiment of FIG. 1, fasteners 105 are shown on the front waist region 102 and are configured to engage with back panels 112 located on the back waist region 101 of disposable garment 100. In other embodiments, a hook(s) may be disposed on the front waist region 102 with a corresponding loop(s) on back waist region 101. In certain embodiments, back panels 112 and fasteners 105 may comprise, respectively, mating parts of a hook and loop fastener: that is, ear portions 104 may comprise loops while fasteners 105 comprise hooks, or vice-versa.

Additionally, absorbent garment 100 may comprise elastic portions to help conform the absorbent diaper to the wearer's body. For example, generally some part of the back waist region 101 comprises elastic material 107 and some part of the front waist region 102 comprises elastic material 111 to fit the garment to the wearer's waist. Also, both sides of the crotch region 103 may comprise elastic material 108 to fit the garment to the wearer's legs, and to provide sealing means to keep any extra liquid or exudate inside of the diaper. Suitable material for use as elastic members are well known to those skilled in the art. An embodiment of elastic material construction includes sheets or strands of ribbons of polymeric, elastomeric material adhered to a sheet in the extended configuration. These elastic members may include polyurethane, synthetic rubber, natural rubber, or any combination thereof. Exemplary embodiments of elastic members that may be used with the present invention are given in U.S. Pat. No. 4,892,536 issued Jan. 9, 1990 to DesMarais et al., U.S. Pat. No. 5,540,796 issued Jul. 30, 1996 to Fries, U.S. Pat. No. 4,895,568 issued Jan. 23, 1990 to Enloe, and U.S. Pat. No. 5,196,000 issued Mar. 23, 1993 to Clear et al. In a preferred embodiment, elastic material 107, 108. May comprise Invista T837 elastic, available from Invista, Wichita, Kans.

The diaper may additionally include stand-up or barrier cuffs or flaps that run along the longitudinal centerline close to the leg edge of the diaper. These cuffs provide extra sealing to keep excess fluids and material inside of the diaper. The cuffs may have additional elastic in them to better fit the wearer, and may also comprise absorbent material to prevent leakage. Exemplary embodiments of cuffs are given in U.S. Pat. No. 4,704,116 issued Nov. 3, 1987 to Enloe, U.S. Pat. No. 5,582,606 issued Dec. 10, 1996 to Bruemmer et al., U.S. Pat. No. 4,795,454 issued Jan. 3, 1989 to Dragoo, and U.S. Pat. No. 5,085,654 issued Feb. 4, 1992 to Buell.

Absorbent garment 100 also includes acquisition distribution layer (ADL) 109. ADL 109 can be a nonwoven sublayer placed between topsheet 106 and absorbent core 110. ADL 109 functions to distribute liquid over a larger surface area in order to increase the overall rate of liquid absorption of absorbent core 110.

The various components of the diaper, e.g., topsheet 106, ADL 109, absorbent core 110, and ear portions 104, etc., are integrally assembled together using various types of suitable attachment means that are well known in the art. These attachments means include the non-limiting examples of adhesives, sonic boding, thermal boding, or any combination thereof.

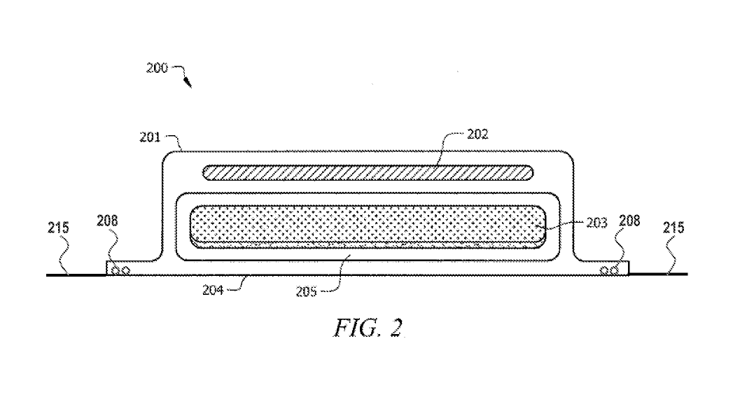

FIG. 2 illustrates a cross-section of an absorbent diaper 200 taken through the crotch region along line 2-2′. This cross-section comprises a topsheet 201, an ADL 202, an absorbent core 203, a backsheet 204, and fasteners 215. Elastic material 208, such as elastic strands, elastic members, elastic ribbons, or other types of elastic are disposed between topsheet 201 and backsheet 204, and can be adhered to one or both of topsheet 201 and backsheet 204. Elastic material 208 is generally located on the diaper 200 at or near its outer edges.

Additionally, as depicted in FIG. 2, diaper 200 may also include a tissue or nonwoven cover 205 above, below or around the absorbent core 203. Nonwoven cover 205 provides containment of the core materials preventing migration of fines and particulates during manufacturing and use and acts as a barrier preventing direct contact of the core and the wearer's skin. Cover 205 may also provide additional structural stability to the core.

Backsheet 204 is generally that portion of diaper 200 positioned adjacent the garment facing surface of absorbent core 203 which prevents the exudates absorbed and contained therein from soiling articles that may contact diaper 200. In preferred embodiments, backsheet 204 is impervious to liquids (e.g., urine) and comprises a thin plastic film such as a thermoplastic film having a thickness of about 0.012 mm (0.5 mils) to about 0.051 mm (2.0 mils). Suitable backsheet films include those manufactured by Tredegar Industries Inc. of Terre Haute, Ind. and sold under the trade name X15306, X10962 and X10964.

Other suitable backsheet materials may include breathable materials which permit vapors to escape from diaper 200 while still preventing exudates from passing through backsheet 204. Exemplary breathable materials may include materials such as woven webs, nonwoven webs, composite materials such as film-coated nonwoven webs, and macroporous films such as manufactured by Mitsui Toatsu Co. of Japan under the designation ESPOIR NO and by Tredegar Corp. of Richmond, Va. under the designation EXAIRE. Suitable breathable composite materials comprising polymer blends are available from Clopay Corporation, Cincinnati, Ohio under the name HYTREL blend P18-3097. In a preferred embodiment, backsheet 204 comprises a 230 mm wide, 30 gsm Providencia nonwoven, which is available from Polymer Group, Inc., Statesville, N.C.

Backsheet 204 may be joined to topsheet 201, absorbent core 203 or any other element of the diaper 200 by any attachment means known in the art. For example, the attachment means may include a uniform continuous layer of adhesive, a patterned layer of adhesive, or an array of separate lines, spirals, or spots of adhesive. One preferred attachment means comprises an open pattern network of filaments of adhesive as disclosed in U.S. Pat. No. 4,573,986 issued Mar. 4, 1986 to Minetola et al. Other suitable attachment means include several lines of adhesive filaments which are swirled into a spiral pattern, as is illustrated by the apparatus and methods shown in U.S. Patent No. 3,911,173 issued Oct. 7, 1975 to Sprague, Jr., U.S. Pat. No. 4,785,996 issued Nov. 22, 1978 to Ziecker, et al., and U.S. Pat. No. 4,842,666 issued Jun. 27, 1989 to Werenicz. Each of these patents are incorporated herein by reference. Adhesives which have been found to be satisfactory are manufactured by H. B. Fuller Company of St. Paul, Minn. and marketed as HL-1620 and HL-1358-XZP. Alternatively, the attachment means may comprise heat bonds, pressure bonds, ultrasonic bonds, dynamic mechanical bonds, or any other suitable attachment means or combinations of these attachment means as are known in the art.

Topsheet 201 is preferably positioned adjacent to the body of a user and between the user and absorbent core 203. Topsheet 201 may be joined to absorbent core 203 and/or to backsheet 204 by any attachment means suitable for such joining, such as discussed above. In some embodiments, topsheet 201 and backsheet 204 are joined directly in some locations and indirectly joined in other locations by directly joining topsheet 201 to other elements of diaper 200.

Topsheet 201 is preferably compliant, soft feeling, and non-irritating to the wearer's skin. Further, at least a portion of topsheet 201 is liquid pervious, permitting liquids to readily penetrate through its thickness. A suitable topsheet 201 may be manufactured from a wide range of materials, such as porous foams, reticulated foams, apertured plastic films, or woven or nonwoven webs of natural fibers (e.g., wood or cotton fibers), synthetic fibers (e.g., polyester or polypropylene fibers), or a combination of natural and synthetic fibers. If the absorbent assemblies include fibers, the fibers may be spunbond, carded, wet-laid, meltblown, hydroentangled, or otherwise processed as is known in the art. For example, one suitable topsheet 201 comprising a web of staple length polypropylene fibers is manufactured by Veratec, Inc., a Division of International Paper Company, of Walpole, Mass. under the designation P-8. Other suitable topsheets may comprise Polypropylene Spunbond-Meltblown-Spunbond (SMS) and Spunbond (SSS) available from from Fitesa (Simpsonville, S.C.), Avgol (Mocksville, N.C.), or Polymer Group Inc. (Charlotte, N.C.).

Preferably, topsheet 201 is made of a hydrophobic material to isolate the wearer's skin from liquids contained in absorbent core 203. If topsheet 201 is made of a hydrophobic material, preferably at least the upper surface of topsheet 201 is treated to be hydrophilic so that liquids will transfer through the topsheet more rapidly. This diminishes the likelihood that body exudates will flow off of topsheet 201 rather than being drawn through topsheet 201 and being absorbed by the absorbent core 203. Topsheet 201 can be rendered hydrophilic by treating it with a surfactant or by incorporating a surfactant into the topsheet. Suitable methods for treating topsheet 201 with a surfactant include spraying the topsheet material with the surfactant and immersing the material into the surfactant. A more detailed discussion of such a treatment and hydrophilicity is contained in U.S. Pat. No. 4,988,344 issued Jan. 29, 1991 to Reising, et al. and U.S. Pat. No. 4,988,345 issued Jan. 29, 1991 to Reising. A more detailed discussion of some suitable methods for incorporating surfactant in the topsheet can be found in U.S. Statutory Invention Registration No. H1670, published on Jul. 1, 1997 in the names of Aziz et al. Alternatively, topsheet 201 may include an apertured web or film that is hydrophobic. This may be accomplished eliminating the hydrophilizing treatment step from the production process and/or applying a hydrophobic treatment to topsheet 201, such as a polytetraflouroethylene compound like SCOTCHGUARD or a hydrophobic lotion composition, as described below. In such embodiments, it is preferred that the apertures be large enough to allow the penetration of aqueous fluids like urine without significant resistance.

Any portion of the topsheet may be coated with a lotion as is known in the art. The lotion may function alone or in combination with another agent as the hydrophobizing treatment described above. Topsheet 201 may also include or be treated with antibacterial agents. Further, topsheet 201, backsheet 204, or any portion of topsheet 201 or backsheet 204 may be embossed and/or matte finished to provide a more cloth like appearance.

The illustrated embodiment of FIG. 2 includes ADL 202. ADL 202 may also be known as a surge management layer. ADL 202 incorporates materials that are designed primarily to receive, temporarily store, and/or distribute liquid along the mutually facing surface with absorbent core 203, thereby increasing the overall rate of liquid absorption of absorbent core 203. One suitable material for ADL 202 comprises a material having a basis weight of about 50 grams per square meter, and comprising a through-air-bonded-carded web of a homogeneous blend of 60% 3 denier bicomponent fiber comprising a polyester core/polyethylene sheath, commercially available from BASF Corporation, and 40% 6 denier polyester fiber. Additionally, ADL 202 may comprise any of or combination of, and not limited to, spun, carded, meltblown, wetlaid, hydroentangled fibers or apertured film.

Conventional absorbent cores 203 may be comprised of a variety of different materials, such as hydrophilic fibers, cellulosic fluff such as wood pulp, superabsorbent hydrogel forming particles, synthetic polymeric, meltblown fibers, natural fibers, or combinations thereof. Example embodiments utilize unbleached pulp in absorbent core 203 and/or a combination of bleached and unbleached pulp. Further, absorbent core 203 may be made from a uniform mixture of materials, or may be non-uniformly mixed.

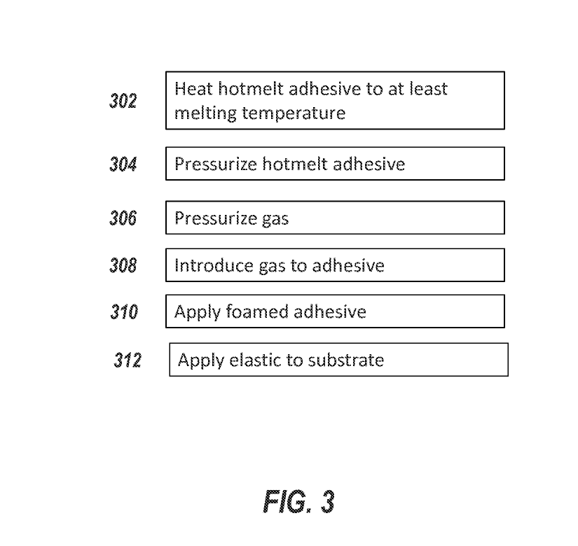

Methods of securing elastic strands using various embodiments of foamed adhesives as discussed below in connection with FIG. 3.

For example, in certain embodiments a hotmelt adhesive may be mixed with a gas, and the resulting foamed adhesive may be applied to secure the elastic strands to a substrate.

Specifically, an apparatus for foaming and applying hotmelt adhesive is coupled to a compressed gas line. Suitable apparatuses for foaming and applying hotmelt adhesives are available from Nordson, Westlake, Ohio, and may include for example the Nordson SureFoam Dispensing System. The compressed gas becomes interspersed with the molten adhesive prior to application. The pressurized mixture exits the apparatus and becomes exposed to ambient pressure, and swells as the pressurized gas expands against ambient pressure. The gas becomes suspended in the adhesive and the adhesive becomes foamed as the expanding gas creates pockets within the adhesive.

In preferred embodiments, the applied volume per unit area of foamed adhesive is the same or about the same as the applied volume per unit area of conventional adhesive. This reduces the total weight of the applied adhesive because the foamed adhesive is less dense than conventional adhesive.

In one example, the hotmelt adhesive comprises Bostik H20043 adhesive, available from Bostik, Inc., Wauwatosa, Wis. and the gas comprises nitrogen gas (N₂). Other suitable hotmelt adhesives may be used in other embodiments. Further, other inert gases besides nitrogen gas may be used in other embodiments, and in still other embodiments, air may be used instead of an inert gas.

The adhesive is heated to a suitable melting temperature, in this example about 320° F., such that the adhesive becomes molten. The adhesive is pressurized to about 440 psi and the nitrogen gas is pressurized to an outlet pressure of about 500 psi, and then the adhesive and gas are mixed, yielding a 50% foamed adhesive.

Changing the pressures of the adhesive and the gas changes the percentage that the adhesive is foamed. Lowering the adhesive pressure and keeping the gas pressure constant yields a foamed adhesive with a higher foamed percentage; similarly, raising the adhesive pressure and keeping the gas pressure constant yields a foamed adhesive with a lower foamed percentage. In various embodiments, the adhesive may be pressurized between about 300 psi and about 600 psi and the gas can be pressurized between about 400 psi and about 600 psi.

Preferably, the foamed adhesive will be between about 30% foamed and about 50% foamed. More preferably, the foamed adhesive will be between about 40% foamed and about 45% foamed.

In some embodiments, the foamed adhesive is then applied to a substrate, such as a backsheet, a topsheet, a leg cuff, a leg gather, or other suitable substrate material, or to the elastic strands. Then, the elastic strands and the substrate are combined such that each is adhered to the other.

In other embodiments, the foamed adhesive can be applied directly to the elastic strands. In other embodiments, the foamed adhesive can be applied to both the elastic strands and the substrate.

The foamed adhesive is less dense than a conventional adhesive but retains the same or about the same adhesive strength. Further, the bubbles and pockets formed within the foamed adhesive increase the total surface area of the adhesive, providing improved securement of the elastic members. In addition, when the foamed adhesive is applied to a substrate (i.e., after it leaves the apparatus), the foamed adhesive expands in the z-direction, which further provides improved securement of the elastic members.

One of ordinary skill in the art will understand that the present invention is also applicable to other disposable absorbent articles and, more particularly, to disposable absorbent garments including training pants and adult incontinence garments.

We claim:

1. A disposable absorbent article comprising:
a topsheet;
a backsheet;
an absorbent core disposed between the topsheet and the backsheet;
a front waist region;
a back waist region;
a crotch region;
a plurality of elastic members adhered to a substrate with a foamed hotmelt adhesive;
where the foamed hotmelt adhesive comprises a hotmelt adhesive mixed with a gas such that the foamed hotmelt adhesive is between about 30% foamed and about 50% foamed.

2. The disposable absorbent article of claim 1, where the foamed hotmelt adhesive is between about 40% foamed and about 45% foamed.

3. The disposable absorbent article of claim 1, where the gas is an inert gas.

4. The disposable absorbent article of claim 3, where the inert gas is nitrogen.

5. The disposable absorbent article of claim 1, where the gas is air.

6. The disposable absorbent article of claim 1, where substrate is the backsheet.

7. The disposable absorbent article of claim 6, where some of the plurality of elastic members are adhered to the backsheet at the crotch region.

8. The disposable absorbent article of claim 6, where some of the plurality of elastic members are adhered to the backsheet at the front waist region.

9. The disposable absorbent article of claim 6, where some of the plurality of elastic members are adhered to the backsheet at the back waist region.

10. The disposable absorbent article of claim 1, further comprising a leg cuff, where the substrate is the leg cuff.

11. A method of securing elastic members to a substrate with foamed hotmelt adhesive, comprising introducing a gas to a molten hotmelt adhesive, and adhering elastic strands to the substrate via the foamed hotmelt adhesive.

12. The method of claim 11, wherein the gas is an inert gas.

13. The method of claim 12, where the inert gas is nitrogen.

14. The method of claim 13, where the gas is air.
